(19) United States
(12) Patent Application Publication
(10) Pub. No.: US 2020/0229944 A1
(43) Pub. Date: Jul. 23, 2020

(54) FOOTPRINT EXPANDING CAGE

(71) Applicant: CTL Medical Corporation, Addison, TX (US)

(72) Inventors: Jon Suh, Ambler, PA (US); Sean SUH, Milltown, NJ (US)

(21) Appl. No.: 16/749,777

(22) Filed: Jan. 22, 2020

Related U.S. Application Data
(60) Provisional application No. 62/795,410, filed on Jan. 22, 2019.

(51) Int. Cl. A61F 2/44

(52) U.S. Cl. CPC A61F 2/4455; A61F 2002/3093

(57) ABSTRACT

Disclosed are devices for the fixation and support of vertebrae, particularly spinal implant devices having adjustability in size, shape and/or configuration.

### CROSS-REFERENCE TO RELATED APPLICATIONS

This application claims the benefit of U.S. Provisional Application No. 62/795,410 entitled “Footprint Expanding Cage,” filed Jan. 22, 2019, the disclosure of which is incorporated by reference herein in its entirety.

### TECHNICAL FIELD

The present subject matter relates generally to devices for the fixation and support of vertebrae. In particular, the present subject matter relates to an implant device having adjustability.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

The spinal curvature disorders and/or contour issues present on the surfaces of the vertebrae may present additional challenges. As such, there is need for further improvement, and the present subject matter is such improvement.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In one exemplary embodiment, the implant system may contain a single or unilateral implant between two vertebrae. Using a unilateral implant may provide for construct stability, minimize access for decompression, and reduce surgical approach-related morbidities related to muscle injury and implant load, as well as less postoperative pain and quicker patient recovery. In various embodiments, the implant geometry may adjustable, allowing the implant to be expanded from a collapsed position for introduction and/or placement into an anatomical space and then expanded to an enlarged condition to desirably increase the effective surface area of the implant.

In another exemplary embodiment, the implant system may contain a pair of bilateral implants that may be positioned between two adjacent vertebrae. Using a unilateral implant may provide for construct stability, minimize access for decompression, and reduce surgical approach-related morbidities related to muscle injury and implant load, as well as less postoperative pain and quicker patient recovery. In various embodiments, the unilateral implant geometry may adjustable, allowing the implant to be expanded from a collapsed position to an expanded position to increase the surface area of the implant.

In one exemplary embodiment, the implant system may comprise a surgical method for insertion of a spinal implant in a reduced cross-section condition, with deployment of the implant accomplished within the intervertebral disc space. The method can comprise: selecting a surgical approach; preparing the vertebral endplates as required by the selected approach, inserting at least one collapsed implant through at least one incision, finalizing location and/or positioning of collapsed implant prior to deployment; rotating and/or displacing the implant to a desired position; adjusting the implant to laterally expand the implant to some larger cross-sectional configuration, such as 30 to 90 degrees between component parts; and filling the intervertebral space and/r implant voids with a filler material to promote arthrodesis; and completing the surgical procedure.

Disclosed is an implant device for the spine, the implant device being desirably sized and configured for location between two adjacent vertebrae, the device comprising a first engagement member and a second engagement member which are “nested” to some degree, the first engagement member and the second engagement member being pivotally connected; and an adjustment mechanism, the adjustment mechanism capable of displacing portions of the first engagement member relative to the second engagement member.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 is a perspective view of one embodiment of a spine;

FIGS. 2A-2B depicts a top view of one embodiment of an implant on a vertebral body using the oblique lumbar interbody fusion (OLIF) and/or the anterior lumbar interbody fusion (ALIF) surgical technique;

FIGS. 3A-3B depicts a top view of one embodiment of an implant on a vertebral body using the direct lateral interbody fusion (DLIF) surgical technique;

FIGS. 4A-4D depicts a top and side view of one embodiment of an implant on a vertebral body using the transforaminal lumbar interbody fusion (TLIF) surgical technique;

FIG. 5 depicts a top view of one embodiment of an implant on a vertebral body using the posterior lumbar interbody fusion (PLIF);

FIGS. 6A-6C depicts a perspective view of one embodiment of an implant range of motion;

FIGS. 7A-7D depicts various views of one embodiment of an implant before and after rotation;

FIGS. 8A-8H depicts various views of one embodiment of a collapsed implant;

FIG. 8I depicts a cross-section view of the collapsed implant of FIGS. 8A-8H;

FIGS. 9A-9H depicts various views of one embodiment of an expanded implant;

FIG. 9I depicts a cross-section view of the expanded implant of FIG. 9H;

FIG. 10 depicts an exploded perspective view of one embodiment of the implant;

FIGS. 11A-11G depicts various views of one embodiment of the left engagement member;

FIG. 11H depicts a cross-section view of the left engagement member of FIG. 11G;

FIGS. 12A-12G depicts various views of one embodiment of the right engagement member;

FIG. 12H depicts a cross-section view of the right engagement member of FIG. 12G;

FIGS. 13A-13G depicts various views of one embodiment of a linkage arm;

FIG. 13H depicts a cross-section view of the linkage arm of FIG. 13G;

FIGS. 14A-14G depicts various views of one embodiment of a clevis; and

FIGS. 15A-15D depicts various views of one embodiment of a drive-shaft.

### DETAILED DESCRIPTION OF THE INVENTION

The present subject matter relates generally to devices for the fixation and support of vertebrae. In particular, the present subject matter relates to implant devices having adjustability. In various embodiments, this adjustability can allow the implant to be movable from a collapsed position to an expanded position, allowing a relatively small implant to be inserted into a targeted anatomical region and then subsequently expanded to increase the surface area of the implant device in contact with various portions of the resected vertebra.

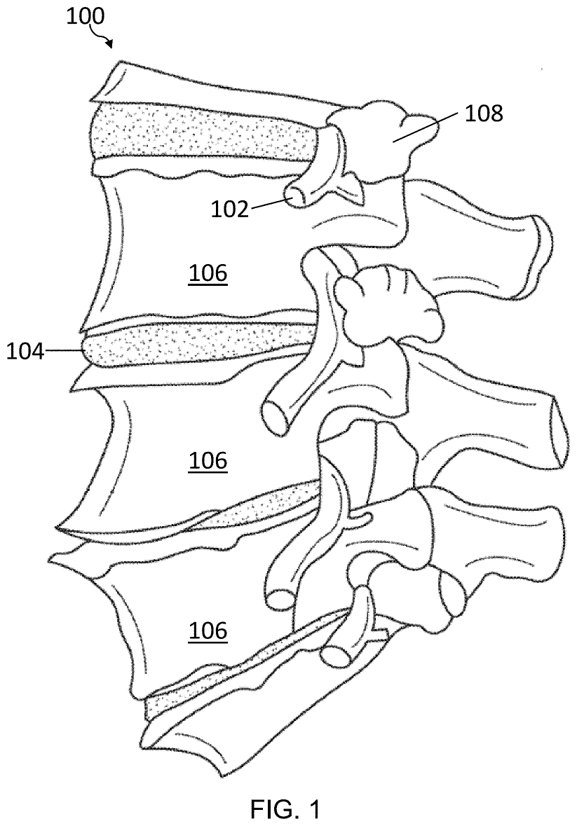

FIG. 1 depicts one embodiment of a portion of a spinal column. The spinal column 100 of vertebrates 106 provides support to bear weight and protection to the delicate spinal cord and spinal nerves 102. The spinal column 100 includes a series of vertebrae 106 stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body 106 is an intervertebral disk 104, a cartilaginous cushion to help absorb impact and dampen compressive forces on the spine. To the posterior, the laminar arch covers the neural structures of the spinal cord and nerves for protection. At the junction of the arch and anterior vertebral body are articulations 108 to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain, and can sustain permanent neurological damage if the conditions are not treated appropriately.

Alternatively or in addition, there are several types of spinal curvature disorders. Examples of such spinal curvature disorders include, but need not be limited to, lordosis, kyphosis and scoliosis.

Aside from the challenges presented by the limited access, size and angulation of various surgical approaches to the spinal anatomy, spinal curvature disorders and/or contour issues can often present on the surfaces of the vertebrae, which may present additional challenges. As such, there is need for further improvement in devices and surgical methods for spinal arthrodesis. The present subject matter is such improvement.

The present subject matter will now be described with reference to the drawings, wherein like reference numerals are used to refer to like elements throughout. It is to be appreciated that the various drawings are not necessarily drawn to scale from one figure to another nor inside a given figure, and in particular that the size of the components are arbitrarily drawn for facilitating the understanding of the drawings. In the following description, for purposes of explanation, numerous specific details are set forth in order to provide a thorough understanding of the present subject matter. It may be evident, however, that the present subject matter can be practiced without these specific details. Additionally, other embodiments of the subject matter are possible and the subject matter is capable of being practiced and carried out in ways other than as described. The terminology and phraseology used in describing the subject matter is employed for the purpose of promoting an understanding of the subject matter and should not be taken as limiting.

Surgical Approaches

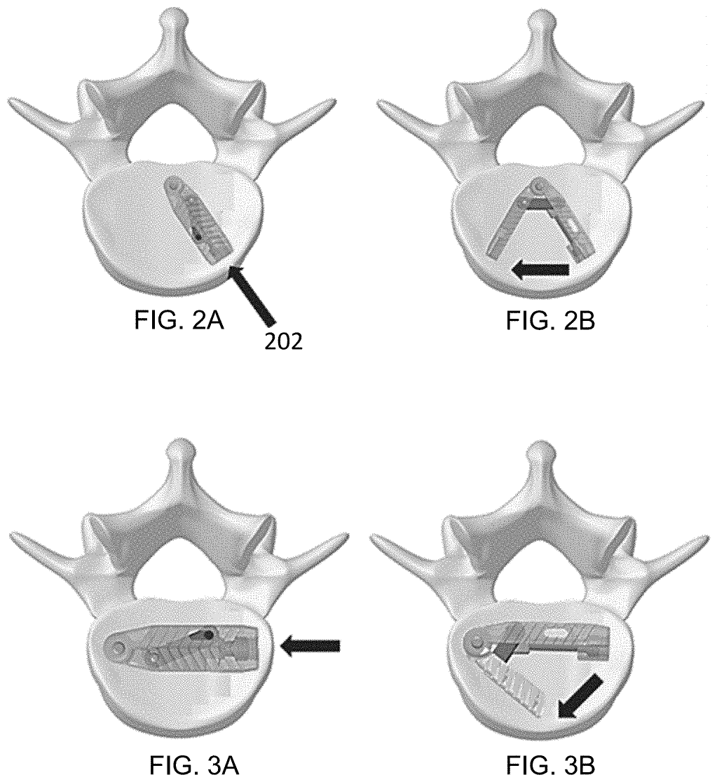

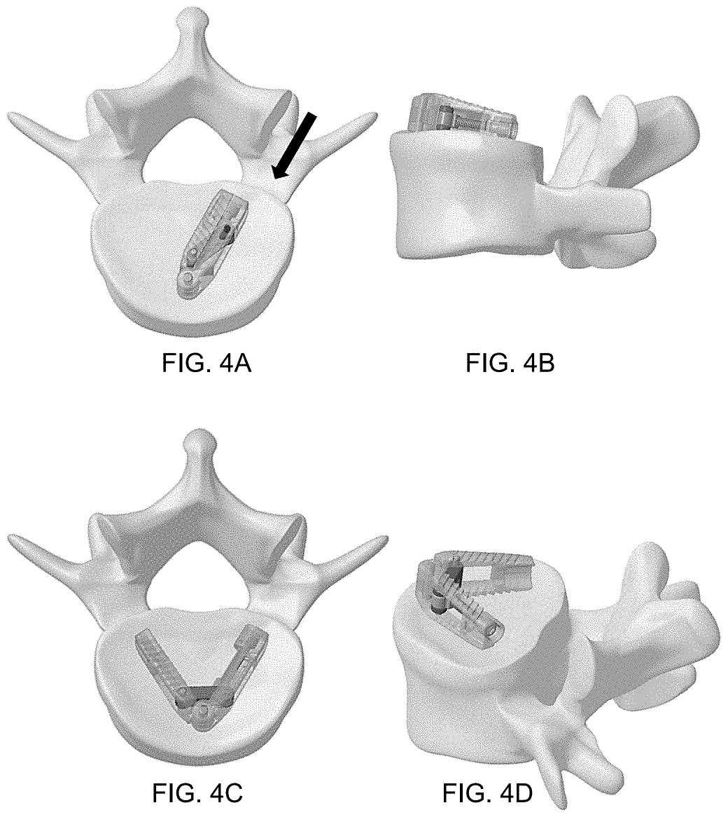

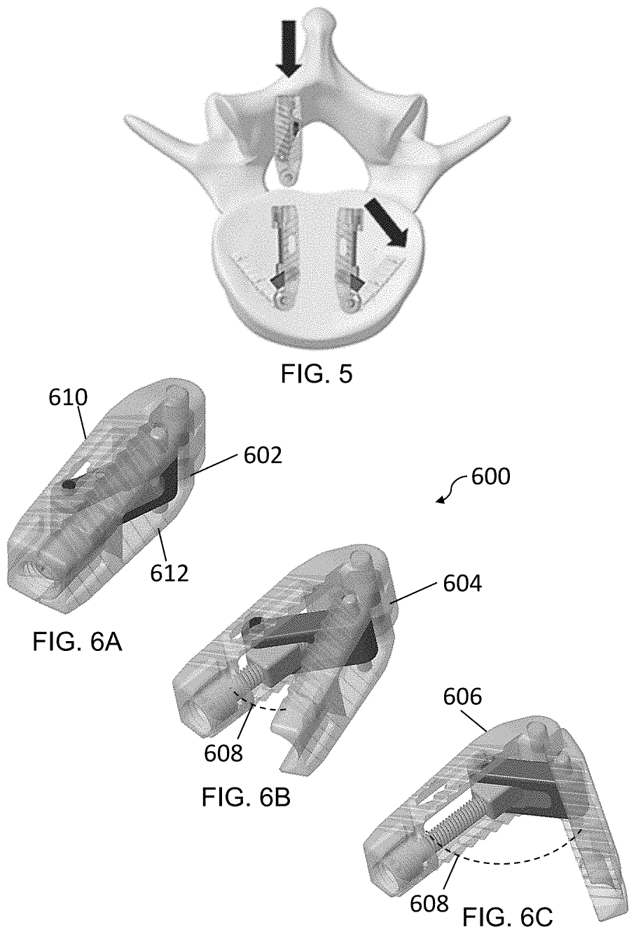

Various of the embodiments of surgical implants described herein may be compatible with a variety of surgical fusion approaches, including posterior lumbar interbody fusion (PLIF) (see FIG. 5), transforaminal lumbar interbody fusion (TLIF or MI-TLIF) (see FIGS. 4A-4D), oblique lumbar interbody fusion/anterior to psoas (OLIF/ATP) (see FIGS. 2A-2B), anterior lumbar interbody fusion (ALIF) (see FIGS. 2A-2B), lateral lumbar interbody fusion (LLIF), direct lateral interbody fusion (DLIF) (see FIGS. 3A-3B), and/or any combinations thereof.

Lumbar interbody fusion (LIF) is an established treatment for a range of spinal disorders. LIF involves placement of an implant (i.e., a cage, spacer or structural graft) within the intervertebral space after discectomy and endplate preparation, and the surgeon typically fills the intervertebral space with a filler material to promote arthrodesis. Such a procedure can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by surgically implanted device(s) to hold the vertebral bodies in proper alignment and desirably allow the bone to heal, much like placing a cast on a fractured bone. Such techniques have been effectively used to treat the above-described conditions and in most cases are effective at reducing the patient's pain and preventing neurological loss of function. As a result, LIF is performed using various approaches, including PLIF, TLIF or MI-TLIF, OLIF/ATP, ALIF, LLIF and/or DLIF. These surgical approaches can also be performed using mini-open or minimally invasive (MIS) techniques.

In one embodiment, the implant system may comprise a surgical method for insertion of a spinal implant in a reduced cross-section condition, with deployment of the implant accomplished within the intervertebral disc space. The method can comprise: selecting a surgical approach; preparing the vertebral endplates as required by the selected approach, inserting at least one collapsed implant through at least one incision, finalizing location and/or positioning of collapsed implant prior to deployment; rotating and/or displacing the implant to a desired position; adjusting the implant to laterally expand the implant to some larger cross-sectional configuration, such as 30 to 90 degrees between component parts; and filling the intervertebral space and/r implant voids with a filler material to promote arthrodesis; and completing the surgical procedure.

In one embodiment, the implant system may comprise a method for insertion and deployment within the intervertebral disc space. The method comprises of: selecting a surgical approach; preparing the vertebral endplates as required by the selected approach, inserting a first collapsed implant through a first incision, finalizing location and/or positioning of first collapsed implant prior to deployment; rotating first collapsed implant 90 degrees or substantially 90 degrees; adjusting the first collapsed implant to laterally expand the right engagement member relative to the left engagement member to a desired angle and increase surface area of the implant; deploy the first expanded implant; inserting a second collapsed implant through a second incision, finalizing location and/or positioning of second collapsed implant prior to deployment and ensuring the second collapsed implant is spaced apart from the first expanded implant; rotating second collapsed implant 90 degrees or substantially 90 degrees; adjusting the second collapsed implant to laterally expand the right engagement member relative to the left engagement member to a desired angle and increase surface area of the implant; deploy the second expanded implant; filling the intervertebral space with a filler material to promote arthrodesis; and completing surgical procedure.

In one embodiment, the collapsed implant may comprise an adjustment mechanism that allows lateral movement of the right engagement member relative to the left engagement member to a desired expansion angle and increase its footprint and/or surface area. FIGS. 6A-6C depict a perspective view of one embodiment of an implant 600 that comprises an adjustment mechanism. The adjustment mechanism may include a screw mechanism that may allow the left engagement member 610 to move laterally relative to the first engagement member 612 to a desired angle 608. The movement may comprise a pivotal movement or sliding movement. The implant 600 may comprise various expansion angles or desired angles 608 during adjustment. The expansion angle or desired angle 608 may comprise a range of 0 degrees to 90 degrees. The expansion angle or desired angle 608 may comprise a range of 0 degrees to 60 degrees. The expansion angle or desired angle 608 may comprise a range of 0 degrees to 45 degrees. The expansion angle or desired angle 608 may comprise a range of 0 degrees to 45 degrees. Alternatively, the expansion angle or desired angle may comprise a range of 0 degrees to 30 degrees or 0 degrees to 15 degrees. The expansion angle or desired angle 608 may comprise collapsed implant 610, a partially expanded implant 604 or a fully expanded implant 606, and/or any combination thereof. The expansion angle or desired angle 608 may reach a partially expanded 604 or fully expanded position 606 using incremental increases, including ½ degree increments, one degree increments, three degree increments, five degree increments, and/or any combinations thereof. In various alternate embodiments, the adjustment mechanism could comprise a jackscrew, a telescoping member, a turnbuckle, a ratchet, or other variable length coupling.

In various embodiments, the screw and/or socket may be fenestrated so that bone graft, marrow, or other therapeutic or structural material may be introduced into the expanded implant center, or implant window.

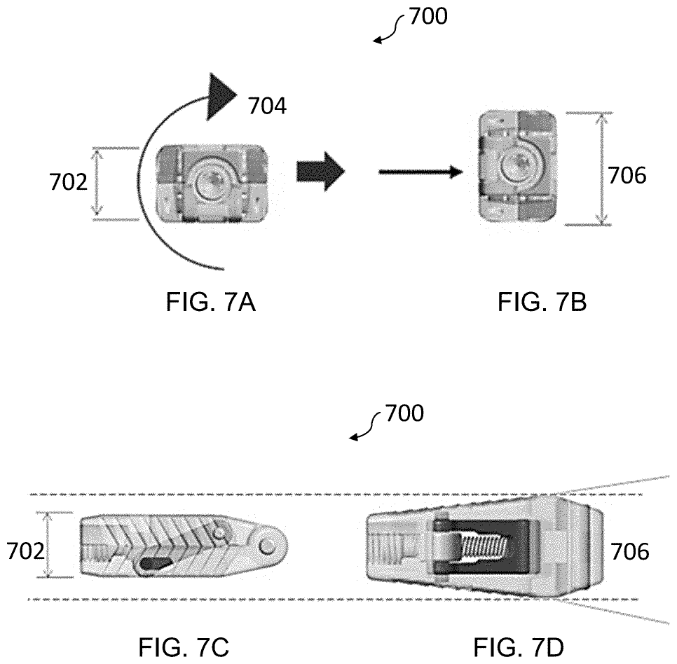

In another embodiment, the collapsed implant allows placement and/or rotation of the implant in situ (i.e., within the intervertebral disc space to restore disc height). FIGS. 7A-7D depict various views of one embodiment of an implant 700 before and after rotation. Once the implant 700 is inserted through the incision, the implant 700 comprises a horizontal height 702. If desired, the implant 700 may be rotated to 90 degrees or substantially 90 degrees with a tool or driver providing for the implant vertical height 706. The implant horizontal height 702 and implant vertical height 706 may comprise a variety of sizes, including small, medium and large. Alternatively, the implant horizontal height 702 and implant vertical height 706 may comprise specific sizes, with a plurality of specific sizes available in a kit form and/or may be customized for each patients' needs.

Implant System

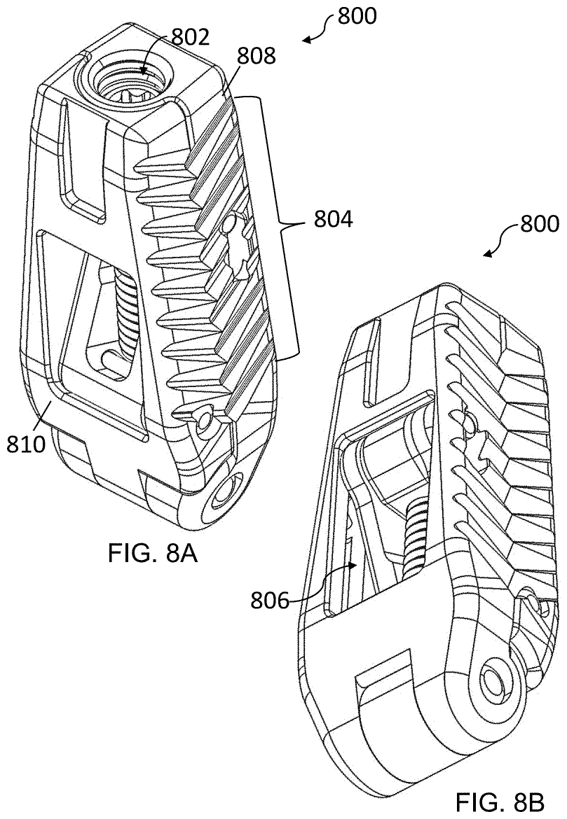

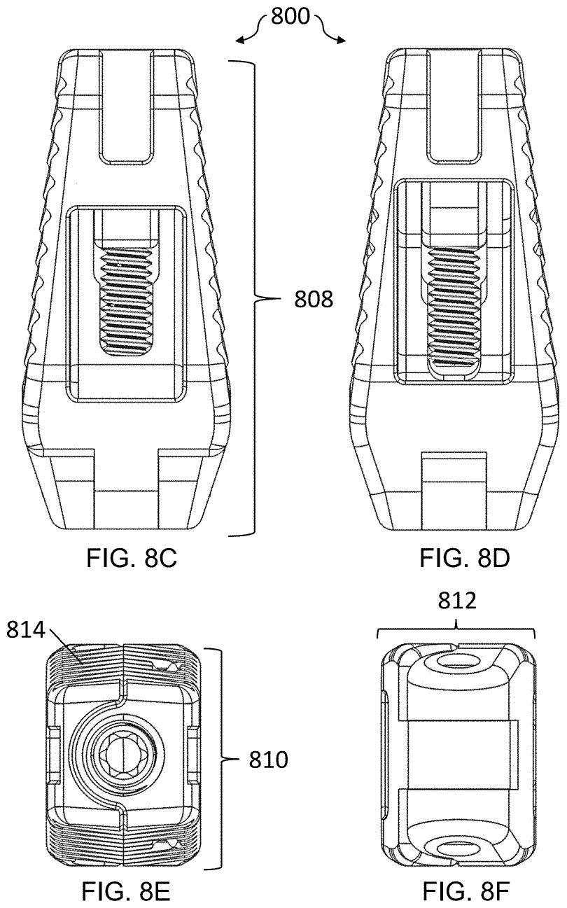

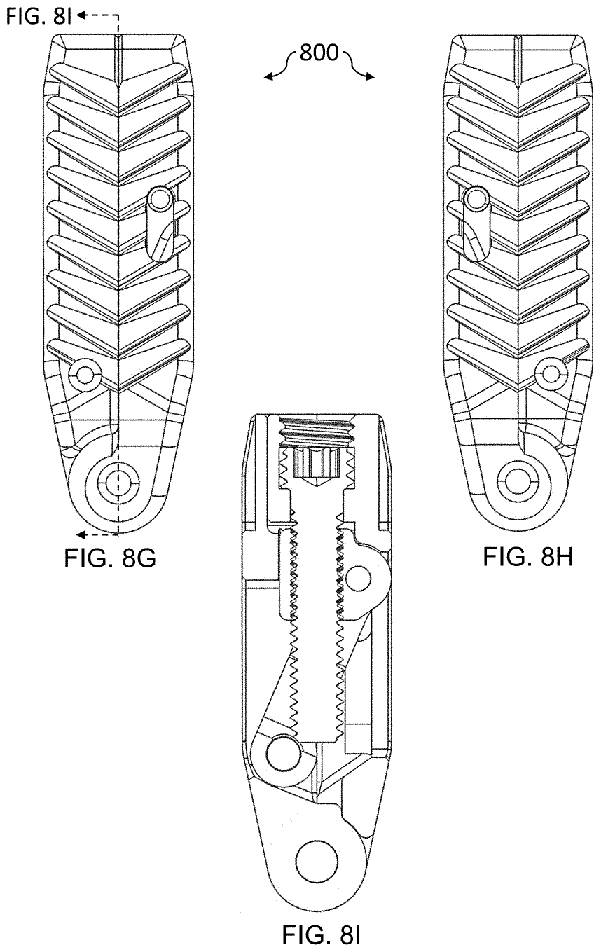

FIGS. 8A-8H depicts various views of one exemplary embodiment of a collapsed implant 800 and FIG. 8I depicts a cross-sectional view of the collapsed implant 800 of FIGS. 8A-8H. The implant 800 may comprise a left engagement member 808, a right engagement member 810, and an adjustment mechanism (not shown). The adjustment mechanism may be positioned to allow the movement of the left engagement member 808 relative to the right engagement member 810 to a desired angle and/or displacement. Alternatively, the adjustment mechanism can be designed to allow the implant 800 to be movable from a collapsed implant position, which desirably allows the implant to be inserted with a minimal cross-section into the intervertebral disc space, and an expanded implant position, which allows the implant to be expanded to a desired angle and/or size within the intervertebral disc space.

In various embodiments, the expanding implant may be formed as a linkage which is movable between a compact configuration and an expanded configuration. A shaft of an adjustment mechanism in the implant may increase and decrease in length to cause the components to move between the compact and expanded configurations, and an implant width may be increased in the expanded configuration. An insertion instrument may releasably grasp the spacer and transform the implant between the compact and expanded configurations.

At least a portion of the adjustment mechanism is disposed between the left engagement member 808 and the right engagement member 810. The adjustment mechanism may comprise a drive shaft, a clevis and a linkage member. The drive shaft is inserted through the passage 802 and into the clevis (not shown). The clevis and the linkage member are connected together at least one end. The drive shaft can be rotated counter-clockwise in an incremental angle to provide an upward force on the clevis, which allows the linkage member to separate the left engagement member 808 relative to the right engagement member 810 to a desired angle. Alternatively, the drive shaft may be rotated clockwise in an incremental manner to provide a downward force on the clevis, which allows the linkage member to close the left engagement member 808 relative to the right engagement member 810 to a collapsed implant position or 0 degrees.

In one embodiment, the implant 800, such as those described and illustrated herein, can comprise a variety of materials, including plastics, metals and ceramics. Metals may include titanium, stainless steel, and cobalt chromium. Plastics may include polyetheretherketone (PEEK) and carbon fiber, and carbon fiber-reinforced PEEK (CFRP). One example that can be used with the present subject matter is PEEK-OPTIMA® polymer (commercially available from Invibio Inc., Greenville, S.C., USA). The PEEK-OPTIMA® polymer is a polyaromatic semicrystalline thermoplastic known generically as polyetheretherketone. The PEEK-OPTIMA® polymer is a biocompatible and inert material. Ceramics may include silicon nitride—it has an excellent biointegration and antimicrobial properties. All materials may be solid or porous. Devices comprising PEEK or similar materials may allow optimal visualization of the spinal column during and after surgery. Interbody devices comprising titanium may provide maximum strength while allowing the maximum volume of bone graft to be incorporated into the device. For example, numerous graft openings may be included in a titanium device while the device still provides the desired support between the vertebral bodies.

Radiolucent materials can be utilized to facilitate radiographic evaluation of fusion material or vertebrae near an implant. For example, radiolucent materials permit x-rays to pass through the implant or components thereof so that developed x-ray pictures provide more visibility of the fusion material and vertebrae without significant interference, such as imaging artifacts, caused by the implant. Radiolucent materials can enable clear visualization through imaging techniques such as x-ray and computer tomography (CT), whereas traditional radiopaque metallic or alloy materials can generate imaging artifacts or scatter that may prevent a comprehensive inspection of the surrounding tissue, vertebra and fusion material. In order to address the general disadvantage that some radiolucent materials lack the strength of radiopaque materials, design modifications may be required to provide adequate structural integrity and durability to the implant device. For example, the thickness of portions of the implant subject to stress and strain can be increased in order to add support and structural integrity. Thicker or bulkier construction can mitigate the stresses of vertebra migration and toggling of the bone fasteners that may cause the implant to bend, crack or otherwise be damaged while in use.

In various embodiments, the implant devices and/or any portions or combination of portions thereof, such as those described and illustrated herein, can be constructed from or incorporate an osteo-inductive and/or osteo-conductive material, such as Silicon Nitride. If desired, some portions of the implant may comprise an intermediate layer of a non-loading bearing material such as morselized bone graft and/or granular or powdered silicon nitride, with load bearing members such as titanium or PEEK positioned inside and/or outside of the non-load bearing members. For example, one or more bone facing surfaces of the disclosed devices could incorporate a surface coating of a silicon nitride material. The disclosed modular implants and/or “cage” structures can also allow for various combinations of materials to be integrated and implanted in a single cage. For example, an outer layer of silicon nitride to promote bony ingrowth may “cover” an inner layer of titanium that provides strength and/or support for the implant. A variety of such component materials could be employed, including metal, plastics and/or ceramics, including (but not limited to) PEEK, titanium, chrome cobalt, allograft, autograft or xenograft bone or other materials, solid Silicon Nitride and/or porous Silicon Nitride, as well as other materials well known in the art.

In various embodiments, the implant 800 may comprise a surface coating or surface roughness to encourage bone ingrowth and adhesion. The surface roughness may include nanometric roughening, heat melting, electron beam melting, alkali melting, a plurality of serrations, and/or any combination thereof. In one example, the implant 800 may have at least one surface comprising a surface roughness 804, 814. The surface roughness 804, 814 can be a plurality of serrations. Accordingly, the implant 800 may comprise a surface coating, the surface coating may include solid titanium, porous titanium, a fiber mesh, plasma surface coatings, sintered beaded coatings, and/or any combination thereof.

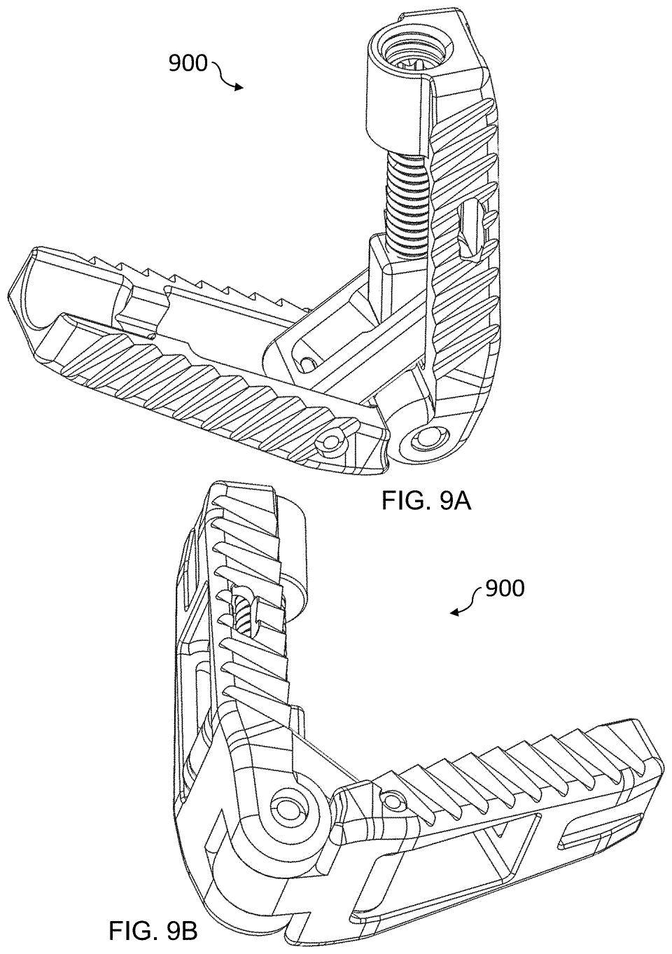

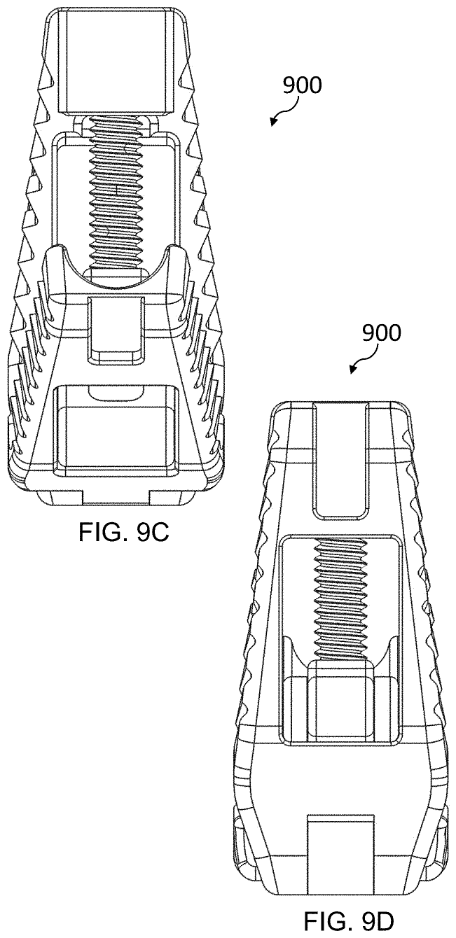

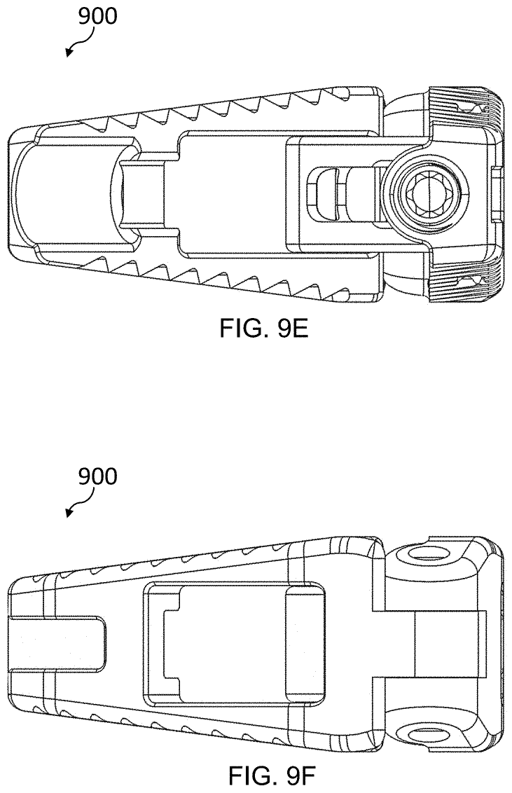

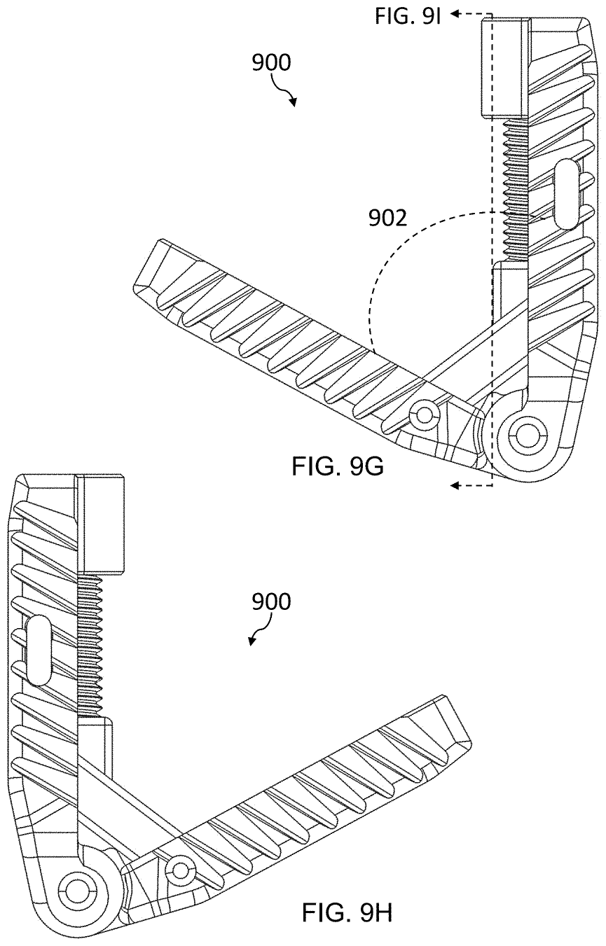

In various embodiments, an implant can have a height 808, a width 810 and a depth 812. The height 808, a width 810 and a depth 812 can be provided in varying sizes in a single kit, including small, medium, large, x-large. Alternatively, the height 808, a width 810 and a depth 812 can be modified to a patient's specific requirements to affect change of the disc height. Also, a plurality of implants can be provided within a kit, which each of the plurality of implants having a height 808, a width 810 and a depth 812 is available within the kit. The plurality of implants implanted into a patient may comprise the same height 808, a width 810 and a depth 812, or the plurality of implants may comprise different heights 808, widths 810 and/or depths 812. FIGS. 9A-9H depicts various views of one embodiment of an expanded implant 900 and FIG. 9I depicts a cross-section view of the expanded implant 900 of FIG. 9H.

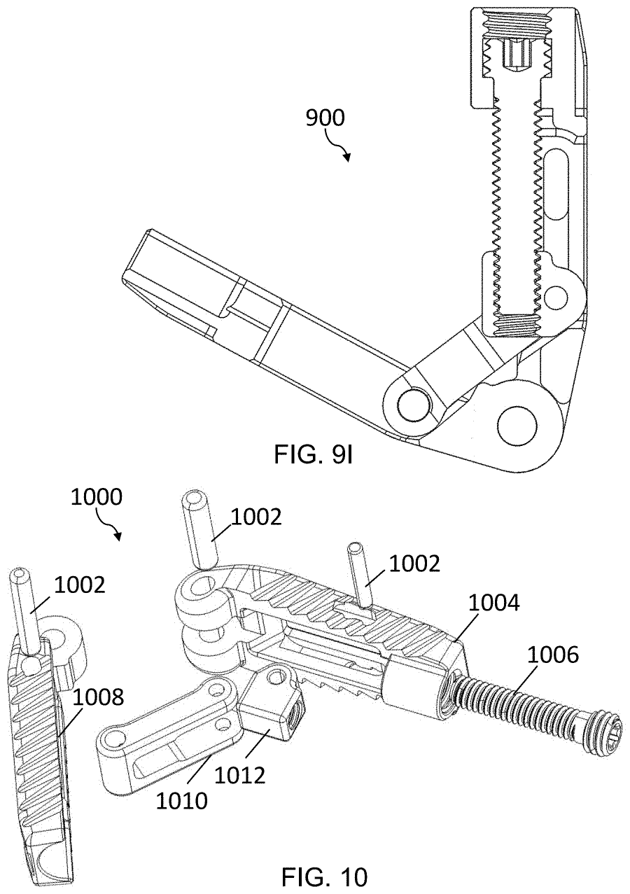

FIG. 10 depicts an exploded view of one embodiment of an implant. The implant 1000 may comprise a left engagement member 1004, a right engagement member 1008, and an adjustment mechanism 1006, 1010, 1012. The adjustment mechanism comprising a drive shaft 1006, a linkage member 1010, and a clevis 1012. At least a portion of the adjustment mechanism 1006, 1010, 1012 is disposed between the left engagement member 1004 and the right engagement member 1008.

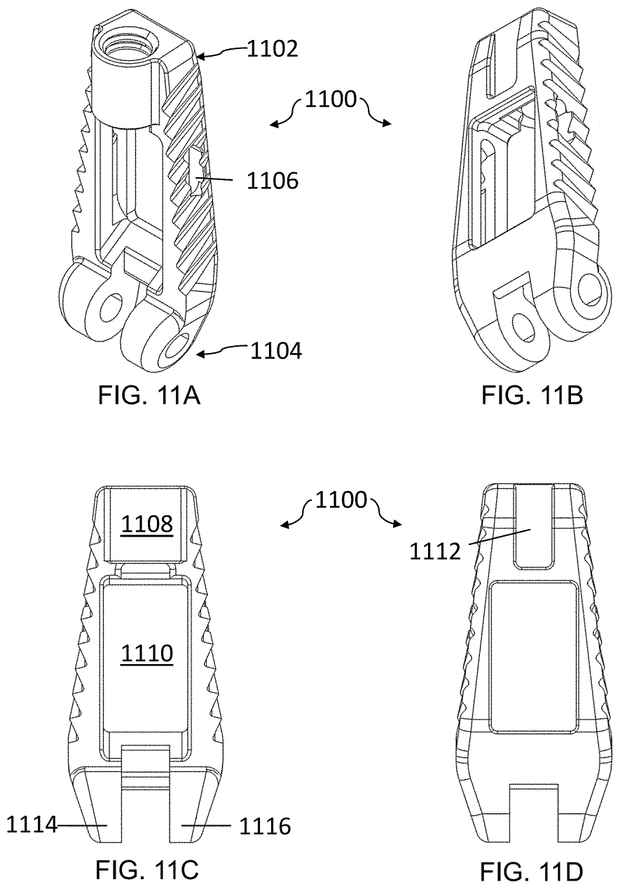

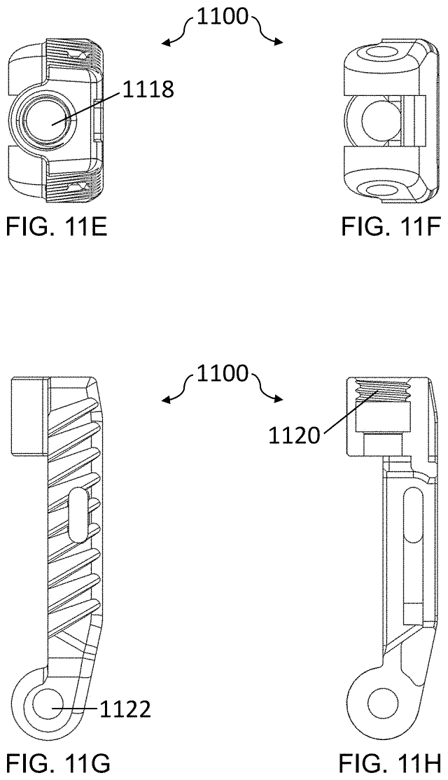

FIGS. 11A-11G depicts various views of one embodiment of the left engagement member 1100. The left engagement member 1100 comprising a first end 1102 and a second end 1104. At least a portion of the second end 1104 including a first leg 1114 and a second leg 1116, the first leg 1114 and the second leg 1116 spaced apart to form a channel. The channel is sized and configured to receive the center leg 1214 from the right engagement member 1200. Each of the first leg 1114, the second leg 1116 and the center leg 1214 having a through-hole 1122, 1218 that extends through each of the legs. The through-holes 1122, 1218 being sized and configured to receive at least one dowel pin (see FIG. 10). The left engagement member 1100 comprising a tapered profile.

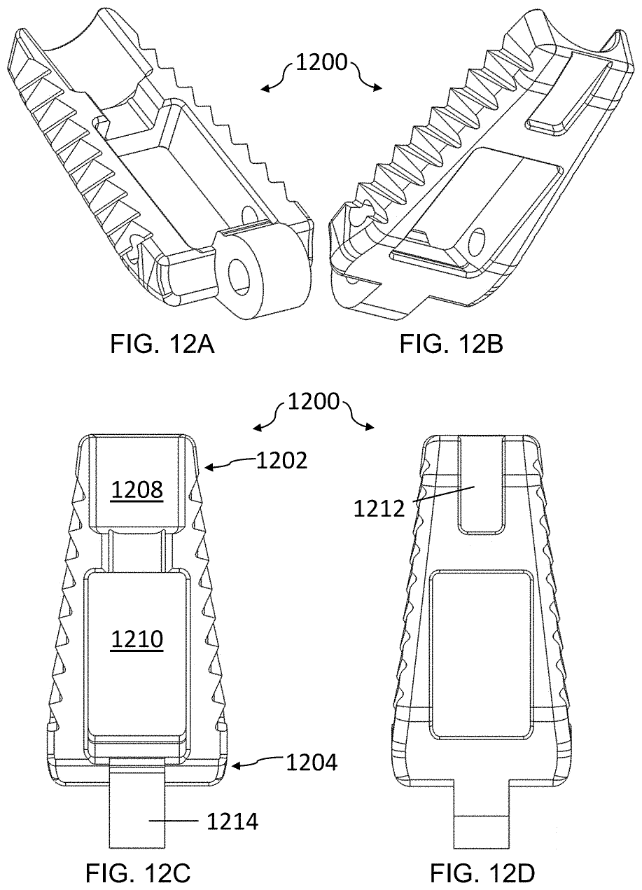

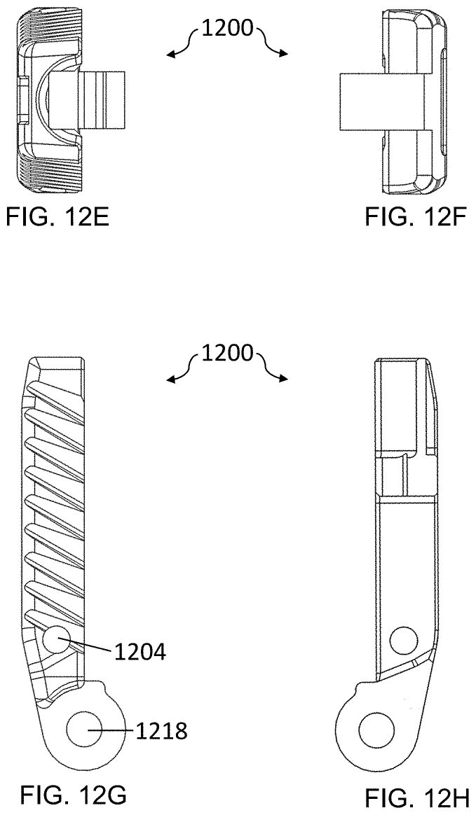

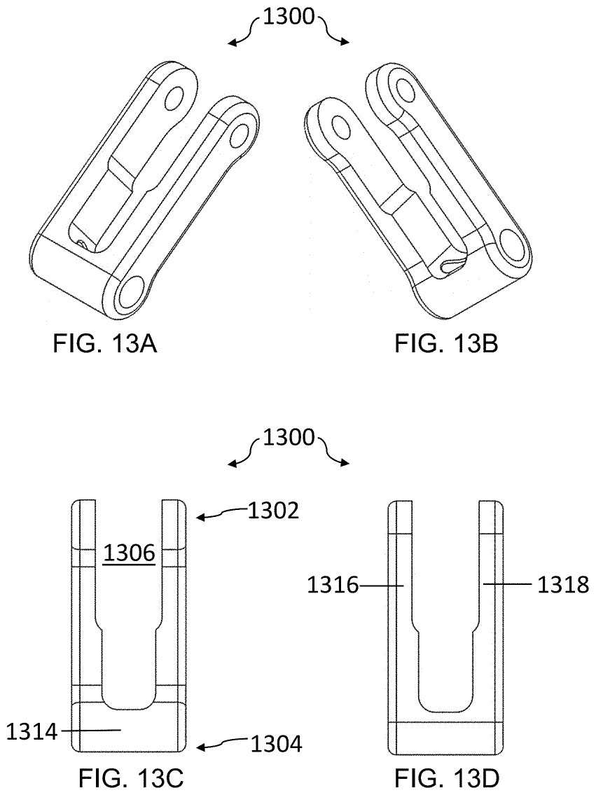

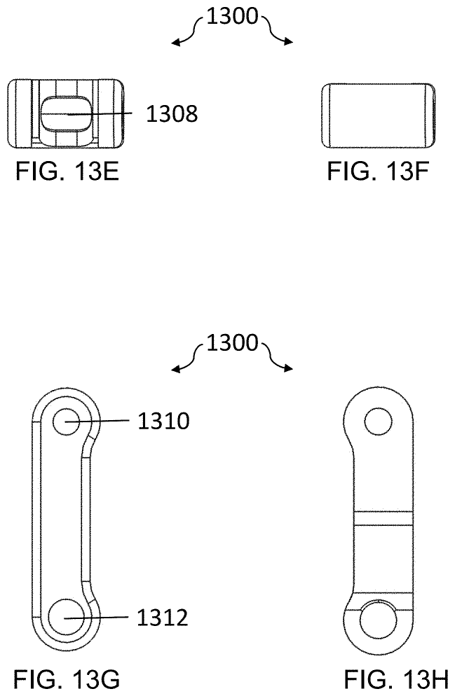

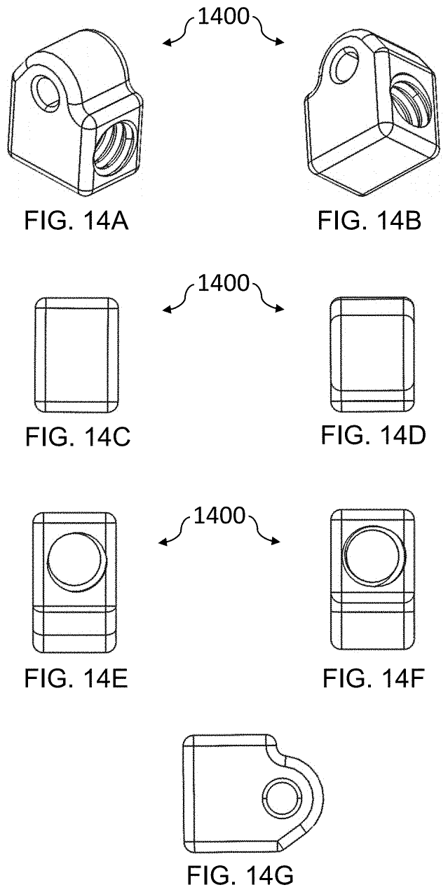

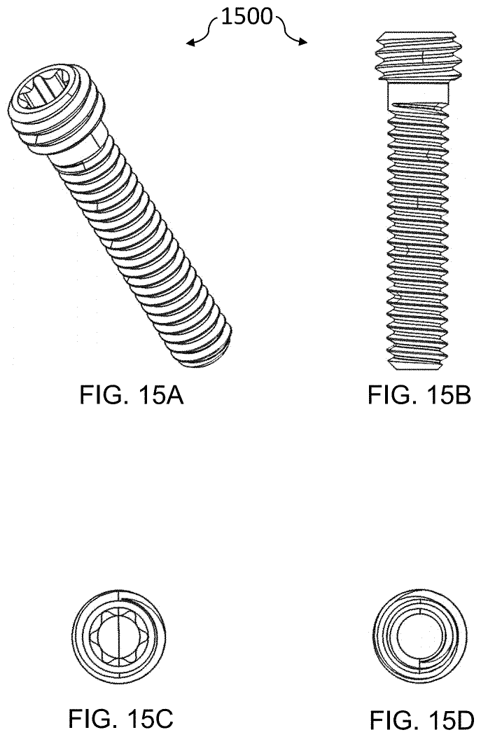

At least one surface of the first end 1102 of the left engagement member 1100 may optionally include a passage 1118. The passage can be sized and configured to receive the drive shaft 1500 (see FIG. 15A-15D). The passage 1118 may be threaded. The first end having a second surface 1108 that protrudes in convex manner or in arch. This second surface 1108 is sized and configured to fit within the recess 1208 of the right engagement member 1200 (see FIGS. 12A-12H). The left engagement member 1100 further comprises an opening 1110 that extends between the first end 1102 and the second end 1104. The opening 1110 being sized and configured to receive at least a portion of the linkage member 1300 (see FIGS. 13A-13G) and the clevis 1400 (see FIGS. 14A-14G). The left engagement member 1100 further comprises a recess 1112. The recess 1112 is disposed or positioned proximate to the first end 1102 or within the first end 1102. The recess 1112 is sized and configured to receive a variety of surgical tools (e.g., clamps, etc.). The recess is disposed an external surface of the left engagement member 1100 proximate to or within the first end 1102. FIG. 11H depicts a cross-section view of the left engagement member of FIG. 11G.

The left engagement member 1100 further comprises a slot 1106. The slot 1106 is sized and configured to receive a dowel pin. The slot 1106 including a length, the length being a travel distance or allotted movement of the left engagement member 1100 relative to the right engagement member 1200. The travel distance is approximately 0.5 mm to 10 mm.

FIGS. 12A-12G depicts various views of one embodiment of the right engagement member 1200. The right engagement member 1200 comprising a first end 1202 and a second end 1204. At least a portion of the second end 1204 including a center leg 1214. The center leg 1214 including a through-hole 1218. The second end 1104 left engagement member 1100 and the second end 1204 of the right engagement member is pivotally connected. Pivotal connection occurs when the center leg 1214 of the right engagement member 1202 is disposed within the channel between the first leg 1114 and a second leg 1116 of the left engagement member. The through-holes 1122, 1218 are colinear or aligned to allow a dowel pin to be inserted and allow a pivotal connection.

The right engagement member 1200 further comprising an opening 1210. The opening 1210 is disposed between the first end 1202 and the second end 1204. The opening 1210 is sized and configured to receive at least a portion of the linkage member 1300 (see FIGS. 13A-13G) and the clevis 1400 (see FIGS. 14A-14G). The right engagement member 1200 further comprises a recess 1212. The recess 1212 is disposed or positioned proximate to the first end 1202 or within the first end 1202. The recess 1212 is sized and configured to receive a variety of surgical tools (e.g., clamps, etc.). The recess 1212 is disposed an external surface of the left engagement member 1200 proximate to or within the first end 1202.

The right engagement member 1200 further comprising a second through-hole 1204. The second through-hole 1204 is sized and configured to receive a dowel pin. At least one end or the second end of the linkage member 1300 (see FIGS. 13A-13G) is pivotally connected to the right engagement member 1200. FIG. 12H depicts a cross-section view of the left engagement member of FIG. 12G.

FIGS. 13A-13G depicts various views of one embodiment of a linkage arm 1300. The linkage arm 1300 having a “U-shaped” body or generally “U-shaped” body. The linkage arm 1300 having a first end 1302 and a second end 1304. Alternatively, the linkage arm 1300 having a base 1314 at the second end 1304, a first arm 1316 and a second arm 1318. The first arm 1316 and the second arm 1318 are spaced apart and separated by a channel 1306. The channel 1306 is sized and configured to receive the clevis 1400 (see FIGS. 14A-14G). The base 1314 including a through-hole 1312, and a recess 1308 that extends from a top surface of the base 1314 towards a portion of a bottom surface of the base 1314. The first arm 1316 and a second arm 1318 extend longitudinally away from the base 1314, and are elongated. The first end 1302 of the first arm 1316 and the second arm 1318 are pivotally connected to the left engagement member 1100. FIG. 13H depicts a cross-section view of the left engagement member of FIG. 13G.

In various embodiments, the right and left engagement members (alternatively referred to as the first and second engagement members) may be regularly or irregularly shaped, and if desired the first engagement member may be shaped as a mirror image of the second engagement member. Desirably, the first engagement member moves relative to the second engagement member in a pivoting motion, and wherein the first engagement member has a bone-contacting surface area greater than a bone-contacting surface area of the second engagement member.

The implant is implantable with a tool, the tool including a tool shaft having a width, and wherein the width of the implant in the compact configuration is about equal to the width of the tool shaft; wherein the tool and/or implant includes an engagement feature that can be selectively operated to grasp the implant and an adjustment feature that can be operated to transform the implant between the compact and the expanded configurations. The right and left engagement members are desirably pivotably joined to each end body at a joint, wherein each joint includes a pin and at least one pin hole.

In various embodiments, one or more of the components of the implant may incorporate textured engagement surfaces that may bear against the respective vertebra. The engagement surface may be textured in any suitable manner, including the use of teeth-like projections. However, it is contemplated that other texturing is possible. For example, the texturing may mimic the texturing of natural bone surface. Such could be accomplished via 3-D material building (e.g., 3-D printing). Metals, such as titanium and stainless steel, or other any other material could be employed for such 3-D material building. One or more bone engagement features such as teeth may project from the bone engagement surfaces. In other embodiments, bone engagement features may include teeth, spikes, pins, posts, points, surface roughening, bosses, ridges, or keels, among others. The size and/or distribution of the bone engagement features may vary.

Desirably, the expandable implant devices disclosed herein can be utilized in a variety of surgical procedures, with the degree of expansion of the implant being selected to accommodate varying desires of the surgeon. For example, where a single implant may be utilized in a fusion procedure (see FIGS. 2A through 4D), it may be desirous for the implant to be expanded to an acute angle, a right angle and/or even an obtuse angle in some embodiments to maximize endplate coverage and promote arthrodesis. Alternatively, where a plurality of implants may be utilized in a fusion procedure (see FIG. 5), it may be desirous for the implant to be expanded to a lesser degree to accommodate the implants and/or maximize endplate support in a desired manner.

Note that with the various figures, different relative positions of the first and second engagement members are shown. In other words, different relative adjustment positions of the first and second engagement members can be accomplished via adjustment in separation and/or surface angulation of portion of one of more of the first and second engagement members to achieve a variety of resulting implant shapes and/or sizes, thereby accommodating virtually any expected anatomical variation. For example, variation of the separation distance between the free ends of the engagement members can desirably cause an increase or decrease in the ultimate size or bone coverage of the implant, due to changes in the positioning of the implant components which engage the adjacent vertebrae. Moreover, various complex combinations (at various amounts) of comparative lateral (e.g., left-right) spacing can be accomplished in one implant, with or without concurrent adjustments in the dimensions of other implants. In various embodiments, each implant can have an engagement area with a different adjusted distance as compared to the other implants, which can be referred to as independent adjustment of the various implant components. Again, if different areas of the vertebrae are chosen/designated for an implant, then the effective endplate coverage for those respective engagement areas can be independently adjusted.

In various exemplary embodiments, a variety of configurations of footprint-expansion intervertebral body spacers with minimal footprint at the time of insertion into a disc space through various anatomical trajectory (ALIF, DLIF, OLIF, PLIF, TLIF) are presented. Unlike typical spinal implants that may damage patient's nerve roots and other structures in the areas near the cage or along the surgical corridor through which they are implanted, the devices of the present invention can allow surgeons to go through a small corridor and change the footprint of the cage to correspond to larger and differing footprints as the patient's spine requires, thereby potentially protecting a patient's nerve roots and other structures in the area proximate to where the cage and/or other devices may pass as they are implanted.

In various alternative embodiments, the implant device may include features to provide height expansion, if desired, or a kit of different height implants may be provided. For example, the implant may be provided with different overall heights covering a range of intervertebral disc heights. In other examples, the implant may be provided with different lordotic and/or kyphotic angles. In still other examples, the implant may be provided with other patterns or features, such as spikes, keels, or the like on the bone contacting surfaces that provide stability and/or resistance to shifting positions. The implant may be made from metal, polymer, ceramic, composite, or other biocompatible and sterilizable material. Different materials may be combined in what is described herein as a single part.

In various embodiments, the disclosed devices can comprise right half and left half hinged or linked cage elements of independent width, length, and heights, connected by a multi-linkage actuator or similar components. If desired, various embodiments may change height, angle of lordosis and/or footprint, or any combinations thereof, when desired by the surgeon.

It is contemplated that size, shape, spacing and/or helix direction of the threaded portions of the adjustment mechanism and/or related components of each implant could be similar or different. For example, the helix direction could be in the same direction or opposite directions. As such, rotating a rotational threaded member of a first implant in a first direction could have the same or different effects as rotating a rotational threaded member of a second implant for adjusting the respective, associated distance between the respective, associated engagement areas of the implants.

In various embodiments, rotation of a threaded adjustment member in a first direction can desirably increase the size of the implant as described herein, while rotation of the threaded adjustment member in an opposing second direction may desirably decrease the size of the implant. This arrangement can allow for retrieval and/or removal of an implant if desired for a variety of reasons. In various alternative embodiments the movement of the adjustment mechanism may include a detent or “one-way” mechanism that allows for expansion but not contraction of the size of the implant, if desired.

Of course, locking/securing mechanisms/means, if desired, are contemplated to help retain the device in a specific adjustment (e.g., at least some distance of the respective engagement areas are adjusted).

In various examples, the implant devices disclosed herein may be utilized to fix and/or secure adjacent vertebrae that have had cartilaginous disc between the vertebrae replaced with fusion material that promotes the fusion of the vertebrae, such as a graft of bone tissue. Also, such can be accomplished even when dealing with a spinal curvature disorder (e.g., lordosis, kyphosis and scoliosis). In addition, method(s) for manufacturing the implant devices and implanting the devices into a spine are contemplated and are part of the scope of the present application.

It should also be understood that the various embodiments of the implant devices described herein may be utilized as stand-alone devices for some surgical applications, and may be utilized in conjunction with various other components (i.e., plates, surgical rods and/or screws, bone graft blocks) in other surgical applications.

In various alternative embodiments, one or more bone-facing surfaces of the implant components described herein could include a textured engagement surface that bears against the respective vertebra, if desired. The engagement surface may be textured in any suitable manner, including teeth-like projections or other texturing. For example, the texturing may mimic the texturing of natural bone surface, or may comprise a bone-ingrowth surface. Such surface features could be accomplished via 3-D material building (e.g., 3-D printing), including the employment of ceramics, plastics and/or metals, such as titanium and stainless steel, or other any other material for such 3-D material building.

Various locking/securing mechanisms/means, if desired, are contemplated to help retain the device and/or components thereof in a specific adjustment (e.g., at least some distance of the respective engagement areas are adjusted). If desired, the implant device may have more or fewer components, and/or the number and distribution of links or other connecting features such as pins may vary accordingly. The devices may include exterior ridges, grooves, teeth, surface roughening, porous coatings or other treatments which enhance fixation to bone and/or bone ingrowth or on growth. The disclosed devices may further include one or more clamps, clips, clasps, braces, snapping mechanisms or other locking devices to hold the device in the compact configuration or in the expanded configuration. Such locking devices may be integral to the interbody device or may be entities separate from the interbody device. The various devices described herein may further include one or more biasing elements to bias the device toward the compact configuration or toward the expanded configuration.

In some embodiments, the device may also include one or more physical stops integrated into the expanding mechanism, the adjustment mechanism, one or more of the engagement members and/or one or more links. The physical stops may limit the transformation of the device into the compact configuration or the expanded configuration, or both. The physical stop or stops may encounter other portions of the device which limit the range of motion of the affected components and thus the expansion/contraction of the related bodies.

In various embodiment, method(s) for manufacturing the disclosed devices and/or implanting the device into a spine are contemplated and are part of the scope of the present application.

The terms “including,” “comprising,” and variations thereof, as used in this disclosure, mean “including, but not limited to,” unless expressly specified otherwise. The terms “a,” “an,” and “the,” as used in this disclosure, means “one or more,” unless expressly specified otherwise.

Devices that are in communication with each other need not be in continuous communication with each other, unless expressly specified otherwise. In addition, devices that are in communication with each other may communicate directly or indirectly through one or more intermediaries.

Although process steps, method steps, or the like, may be described in a sequential order, such processes and methods may be configured to work in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications or modifications of the disclosure. While embodiments and applications of the present subject matter have been shown and described, it should be apparent to those skilled in the art that many more modifications are possible without departing from the inventive concepts herein. The subject matter, therefore, is not to be restricted except in the spirit of the appended claims.

1. An expandable implant device for the spine, the expandable implant device for location between a superior and an inferior vertebrae, comprising:
a first engagement member having a first upper surface for engaging with a lower endplate of the superior vertebra and a first lower surface for engaging with an upper endplate of the inferior vertebra, the first engagement member having a first cavity;
a second engagement member having a second upper surface for engaging with the lower endplate of the superior vertebra and a second lower surface for engaging with the upper endplate of the inferior vertebra, the second engagement member sized to substantially fit within the first cavity;
the first engagement member and the second engagement member being at least partially nested together, the first engagement member being pivotally connected to the second engagement member; and
an adjustment mechanism connected to the first and second engagement members, the adjustment mechanism capable of increasing in length from a first length to a second length to adjust a pivot angle between the first and second engagement members, wherein at the first length the first and second engagement members are substantially parallel and at the second length the first and second engagement members are non-parallel.

2. The implant of claim 1, wherein the adjustment mechanism has a first and a second end, and the first end of the adjustment mechanism is coupled to the first engagement member and the second end of the adjustment mechanism is coupled to the second engagement member.

3. The implant of claim 1, wherein the adjustment mechanism is coupled to the first engagement member by at least one of the group consisting of a rotatable coupling, a pivotal coupling and a slidable coupling.

4. The implant of claim 1, wherein the adjustment mechanism comprises a drive shaft, a clevis and a linkage member.

5. The implant of claim 1, wherein adjusting the expansion angle can alter a pivot angle between the first and second engagement members from 0 degrees to 60 degrees.

6. The implant of claim 1, wherein at least one of the first lower and upper surfaces comprises textured surfaces.

7. The implant of claim 1, wherein at least one of the first lower and upper surfaces comprise silicon nitride.

8. The implant of claim 1, wherein the second engagement member nests completely within the first engagement member when the first and second engagement members are in the substantially parallel configuration.

9. The implant of claim 1, wherein the adjustment mechanism nests completely within the second engagement member when the first and second engagement members are in the substantially parallel configuration.

10. An implant device for the spine, the implant device for location between two adjacent vertebrae, comprising:
a first engagement member, the first engagement member having a first end and a second end;
a second engagement member, the second engagement member having a first end and a second end, the first engagement member second end and the second engagement member second end being pivotally connected; and
an adjustment mechanism, the adjustment mechanism being movable between a collapsed position which prohibits the first and second engagement member from expanding and an expanded position which allows the first engagement member to expand relative to the second engagement member.

11. The implant device of claim 10, wherein the adjustment mechanism is pivotally connected proximate to the first end of the first engagement member and the second end of the second engagement member.

12. The implant device of claim 10, wherein at least one surface of the first and second engagement members comprises silicon nitride.

13. The implant device of claim 10, wherein at least one surface of the first and second engagement members comprises a textured surface.

14. The implant of claim 1, wherein at least one of the first lower and upper surfaces comprise silicon nitride.

15. An intervertebral system, comprising:
an interbody device comprising a plurality of pivotally linked bodies, the interbody device transformable between a compact configuration and an expanded configuration, the interbody device comprising a first elongated engagement member pivotally connected to a second elongated engagement member, the first elongated engagement member including a first channel extending inward from an exterior surface of the first elongated engagement member, the first channel sized and configured to contain the second elongated engagement member;
an adjustment mechanism connected to the first and second elongated engagement members, the adjustment mechanism comprising a rotatable screw connected to the first elongated engagement member and a clevis attached to the second elongated engagement member, wherein rotation of the screw displaces the clevis to increase the length of the adjustment mechanism from a first length to a second length to cause a change in the pivot angle between the first and second elongated engagement members, wherein at the first length the first and second engagement members are substantially parallel and at the second length the first and second engagement members are substantially non-parallel; and
an inserter comprising a shaft portion, an engagement clamp and a rotatable adjustment shaft, the engagement clamp selectively operable to rigidly attach the inserter to the interbody device and the rotatable adjustment shaft operable to transform the implant between a first compact configuration and a second expanded configuration; wherein when the engagement clamp of the inserter is attached to the interbody device, at least a portion of the rotatable adjustment shaft engages with the rotatable screw.

16. The implant of claim 15, wherein the adjustment mechanism is coupled to the first engagement member by at least one of the group consisting of a rotatable coupling, a pivotal coupling and a slidable coupling.

17. The implant of claim 15, wherein the second engagement member nests completely within the first engagement member when the first and second engagement members are in the substantially parallel configuration.

18. The implant of claim 15, wherein the adjustment mechanism nests completely within the second engagement member when the first and second engagement members are in the substantially parallel configuration.

19. The implant device of claim 15, wherein at least one surface of the first and second elongated engagement members comprises silicon nitride.

20. The implant device of claim 15, wherein at least one surface of the first and second elongated engagement members comprises a textured surface.
